(19) United States
(12) Patent Application Publication
(10) Pub. No.: US 2018/0177647 A1
(43) Pub. Date: Jun. 28, 2018

(54) Feminine Hygiene Apparatus

(71) Applicant: La Rena Gonzales, Terra Bella, CA (US)

(72) Inventor: La Rena Gonzales, Terra Bella, CA (US)

(21) Appl. No.: 15/392,627

(22) Filed: Dec. 28, 2016

(51) Int. Cl. A61F 13/53; A61F 13/20; A61F 13/551

(52) U.S. Cl. CPC A61F 13/53; A61F 13/2022; A61F 2013/530802; A61F 13/55175; A61F 13/202

(57) ABSTRACT

This invention relates to a feminine hygiene apparatus, system, and kit that is made of natural absorbent material for the absorption of uterine fluid. The natural absorbent material is has a substantially parallelepiped geometry and configured into a truncated hexahedron shape. The apparatus could be positioned in pelvic cavity below the opening of cervix on both left and right side of vaginal wall into the vaginal fornices. The apparatus could be cleaned, sterilized and reused. Moreover, the apparatus does not produce irritations and discomfort during use as well as it permits sexual intercourse while being present in the vaginal canal.

### (A) CROSS REFERENCE TO RELATED APPLICATIONS

Not applicable

### (B) FEDERALLY SPONSORED RESEARCH AND DEVELOPMENT

Not applicable.

### (C) MICROFICHE

Not applicable

### (D) BACKGROUND OF THE INVENTION

Feminine hygiene products are personal care products used by women during menstruation or uterine/vaginal discharge. Uterine fluid discharge is usually related to pregnancy or its complications, pelvic inflammatory diseases, urinary incontinence, or menstruation between menarche to menopause. Different types of absorption products like sanitary napkins, sanitary towels, tampons, menstrual cups, panty liners, and feminine wipes have been used by females for the absorption of uterine fluid discharge.

A number of different types of feminine hygiene products are available. For example, see: U.S. patent application Ser. No. 10/149,877 issued to Sybille Fuchs et al. discloses a digital vaginal tampon comprising a substantially cylindrical mass of compressed fibers, the tampon being capable of radially expanding into a non-circular shape upon exposure to a wet environment; U.S. patent application Ser. No. 11/890,011 issued to Playtex Products Inc. related to a tampon pledget having an absorbent mass of material and a withdrawal string located at one end. The tampon pledget is initially cylindrical in shape. During use and upon contact with menses or other body fluids, the tampon pledget expands to take on a geometric configuration that approximates the shape and configuration of the vagina into which the tampon pledget is inserted; U.S. patent application Ser. No. 11/787,816 discloses a compressed tampon which comprises a pledget. The pledget when uncompressed has a length, a width and two side edges, and wherein the pledget comprises at least one physical discontinuity.

There are a variety of known problems with current feminine hygiene products. First, they are prone to causing discomfort and irritation to the user during use. Also, the composition of materials as well as their designs can cause medical problems from long term use such as product material allergies, cervical cancer, a deadly condition known as toxic shock syndrome, urinary tract infections, pelvic inflammatory disease, vaginal drying, and yeast infections to name a few. Further most feminine hygiene products are not reusable. Last, feminine hygiene products inserted into the vagina do not allow for sexual intercourse. There is a need for a solution.

### (F) SUMMARY

The present invention is directed to feminine hygiene apparatus that satisfies this need. A feminine hygiene apparatus having features of the present invention comprise a natural absorbent material with a substantially parallelepiped geometry and the shape of a truncated hexahedron. The apparatus is configured to be positioned inside the vaginal cavity into the vaginal fornices, which is located on either side of the cervical opening into the vagina.

An object of the invention is to provide a feminine hygiene apparatus that can absorb uterine fluid completely with substantially no scope of leakage.

An object of the invention is to provide a feminine hygiene apparatus that does not cause irritation or discomfort to the user.

It is further an object of the present invention to provide a feminine hygiene apparatus that permits sexual intercourse while the apparatus is present in the vaginal canal.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

These together with other objects of the invention, along with the various features of novelty which characterize the invention, are pointed out with particularity in the disclosure. For a better understanding of the invention, its operating advantages and the specific objects attained by its uses, reference should be had to the accompanying drawings and descriptive matter in which there are illustrated preferred embodiments of the invention.

### (G) BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 shows a perspective view of an embodiment of the invention.

FIG. 2 shows a top internal view of the pelvic cavity with the embodiment of the apparatus.

FIG. 3 shows a side view of the pelvic cavity with the embodiment of the apparatus.

FIG. 4 shows a top internal view of the pelvic cavity with an alternative embodiment of the apparatus.

### (H) DETAILED DESCRIPTION OF THE INVENTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural and logical changes may be made without departing from the spirit and scope of the present invention. The following detailed description is, therefore, not to be taken in a limiting sense, and the scope of the present invention is defined by the appended claims.

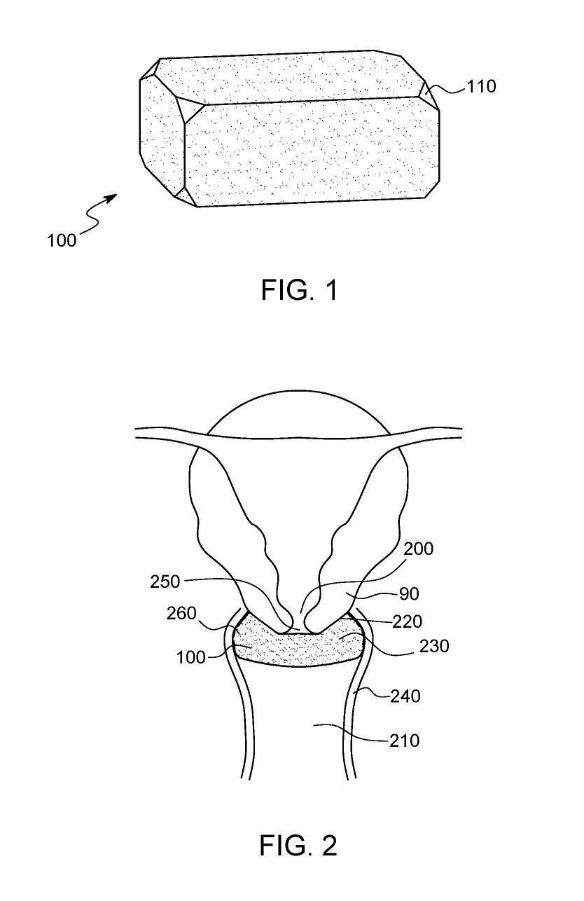

Referring to FIG. 1, an embodiment of the present feminine hygiene apparatus 100 is shown. The apparatus 100 having a parallelepiped geometry and configured into a truncated rectangular prism shape. It is contemplated the apparatus could be any truncated hexahedron shape. The triangular faces 110 are substantially flat and preferably substantially identical in size and angle in relation to the three adjacent surface faces to each triangular face 110. The preferred angle is 45 degrees, but any desired angle is contemplated. Factors affecting the choice of angle could be aesthetic appearance or to increase triangular faces 110 surface area to increase the rate of fluid absorbency, increase comfort for the user, or to increase the ability of apparatus 100 to adhere to a vaginal wall and cervix. Further, it is contemplated individual triangular surfaces 110 within an apparatus may be varied in size and angle for the same reasons stated above and the surface 110 may also include texture if desired to improve at least comfort and ease of use.

Apparatus 100 comprises at least one type of naturally occurring absorbent material such as plant material or sea sponge material. Preferred species of sea sponge materials include Caribbean silk sea sponge, Jade sea sponge, Pearl sea sponge, Sheep's wool sea sponge, and Bahama silk sea sponge. Further, the porosity and absorbency properties of the naturally occurring material are generally consistent throughout the apparatus but may change slightly from time to time due to the inherent variability in using naturally occurring materials. It is contemplated the absorbent material will be free from treatment with pesticides or synthetic chemicals. Further, the density of the material used is as consistent and uniform as possible throughout, but may vary depending on the species of sea sponge material used.

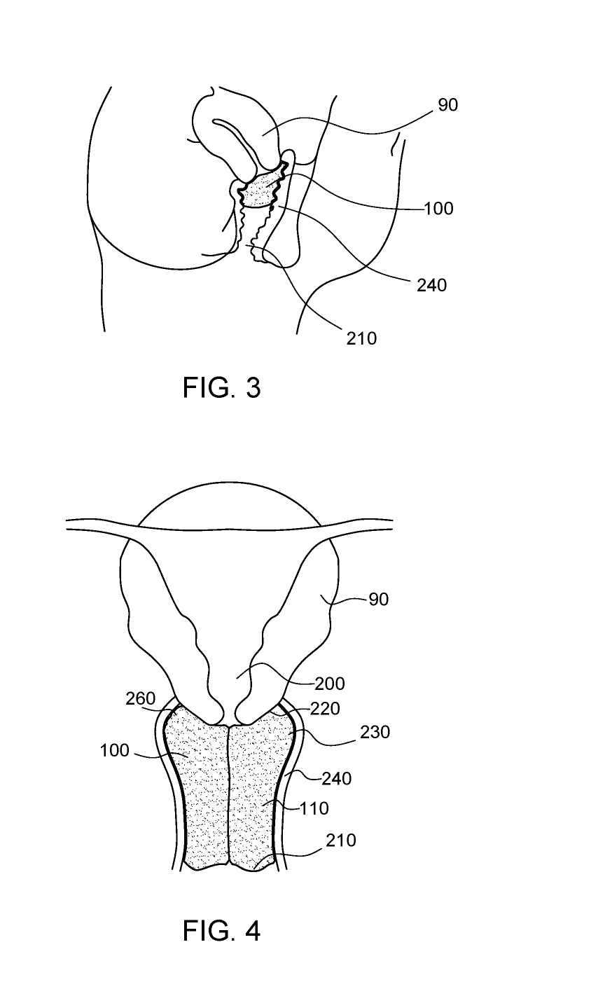

Referring to FIG. 2 and FIG. 3, placement and orientation of an embodiment of the feminine hygiene apparatus 100 at the intersection of the cervix 90 and the vaginal canal 210 is shown. In a preferred embodiment the apparatus 100 is first moistened throughout with fluid comprising at least water before being placed inside the vaginal cavity 210, specifically into at least one vaginal fornix 230 and 260. The moistened apparatus 100 is then formed to temporarily adhere or stick to at least the ectocervix 220, vagina wall 240, and endocervix 250 at the opening to the cervix 200. Further, moistening the apparatus 100 before insertion helps prevent drying which can occur with the use of feminine hygiene products.

The apparatus 100 can be placed and formed in any orientation comfortable to the user, so as long as the placement of all apparatuses creates a substantially complete barrier to uterine fluid, such as menses, from the cervix. The preferred dimensions of apparatus 100 are 2.25 inches in length, 1 inches in width, 1 inches in height, and the length of each side of triangular corner 110 segment being 0.25 inches. These dimensions take into account the dimensions of averages of vaginal width of 1.9 inches to 2.5 inches, and average introital diameter of 1 inch to 2.5 inches, found in Caucasian women. It is contemplated the dimensions of the apparatus can be increased or decreased for accommodate a range of sizes of vaginal fornix, vaginal width, vaginal length or depth, and vaginal introital diameter. Further, multiple apparatuses may be used to achieve the desired results.

Referring to FIG. 4, a feminine hygiene system is shown, comprising at least a first apparatus 100 and second apparatus 110. Each apparatus being formed manually by a user within at least each vaginal fornix 230, 260, and the vaginal wall 240, to ensure substantially complete coverage and blockage of the flow of menses or other uterine fluid from the opening of the cervix 200 into the vaginal canal 210 and ultimately onto a woman's undergarments and clothing.

It is contemplated multiple apparatuses may be used if the user experiences a large volume of uterine or menses flow. Each apparatus 100 and 110 may each be placed into one of the two vaginal fornix 230 and 260. It is further contemplated the multiple apparatuses 100 and 110 maybe by configured horizontally so that apparatus 100 is placed into both vaginal fornix 230 and 260, while apparatus 110 is located below apparatus 100.

The apparatus is left formed in place, keeping the user feeling fresh and dry, until the user desires to manually remove it or the user feels discomfort common with the use of feminine hygiene products in general. A user may manually remove the apparatus when it has reached its' maximum absorbency, which is depends on each individual user and the amount of menses their body releases during a given menstruation cycle.

As with feminine hygiene products made from natural sources, it is important the apparatus is kept fresh, clean, and sterile before use. To ensure all of these criteria are met, one or more apparatuses may be packaged in clean sterile plastic contains and vacuum sealed. It is contemplated the apparatus may be packaged in any other way convenient for manufacturing, distribution, or known in the art for feminine hygiene products.

Removal of the the apparatus is easy and convenient. The apparatus can be removed from the vaginal canal by doing exercises known to strengthen the pelvic floor, such as Kegel exercises, while sitting comfortably on a surface or manually removing each apparatus from the vagina using one hand. Further, a combination of manual removal and exercises may be employed if desired.

The feminine hygiene apparatus of present invention has a unique and novel feature which allows the vaginal canal to be free of obstructions and menses until the apparatus has reached its absorption capacity. This feature also allows for sexual intercourse while the apparatus is in use. Further, the absorption capacity is substantially the same throughout all regions of the apparatus since there are no cores or layers to change the absorption capacity at different regions of the apparatus. Another benefit of the apparatus is that it contours to each individual's specific vaginal and cervical cavity shape without discontinuities to allow uterine fluid leakage. Further the invention is void of any uncomfortable protrusions which can provide discomfort to the user. If there is any discomfort or flow of uterine fluid from the vagina, the user can change the apparatus.

The apparatus can also be cleaned and reused. Cleaning of the feminine hygiene apparatus of the present invention could be carried out at least by thoroughly and repeatedly rinsing the apparatus until all signs of menses are removed, followed by soaking the apparatus in a cleaning solution, followed by soaking the apparatus in a sterilizing solution, and finally followed by drying. After the apparatus is dry it may be stored in cool and dry place. The cleaning and sterilizing solution may include any commonly used method known in the art for cleaning naturally occurring absorbent material.

The apparatus may also be sold in a bulk format without being first washed and sterilized. The apparatus may also be packaged and sold as a kit in a vacuum sealed container before or after sterilization. It is contemplated at least two fresh, clean, and sterile apparatuses would be vacuum sealed inside a container such as a bag or other commonly known vacuum sealing containers in the art, and instructions either printed directly on the container, on a label which is then applied to the container, or a piece of paper or card stock accompanying the container. The instructions include at least information on how to use the apparatus, but could also contain information on how to clean and care for the apparatus as well.

The present invention provides numerous advantages over the traditional methods of feminine hygiene product. The present invention could be used by menstruating females of any age. The apparatus does not produce irritation and discomfort to the female body due to insertion, use, and removal as is common with other feminine hygiene products. Furthermore, the present invention permits sexual intercourse while the apparatus is in place inside the vaginal canal. In addition, the apparatus can be used to absorb uterine fluid for health issues such as urinary incontinence.

It is to be understood that the above description is intended to be illustrative, and not restrictive. For example, the above-discussed embodiments may be used in combination with each other. Many other embodiments will be apparent to those of skill in the art upon reviewing the above description.

The benefits and advantages which may be provided by the present invention have been described above with regard to specific embodiments. These benefits and advantages, and any elements or limitations that may cause them to occur or to become more pronounced are not to be construed as critical, required, or essential features of any or all of the embodiments.

While the present invention has been described with reference to particular embodiments, it should be understood that the embodiments are illustrative and that the scope of the invention is not limited to these embodiments. Many variations, modifications, additions and improvements to the embodiments described above are possible. It is contemplated that these variations, modifications, additions and improvements fall within the scope of the invention.

What is claimed is:

1. A feminine hygiene apparatus comprising:
a natural absorbent material capable of absorbing uterine fluid of a user, wherein said absorbent material is configured to position into a vaginal fornix; and
said absorbent material is configured to adhere to the vaginal wall around said vaginal fornix; and
said absorbent material having substantially parallelepiped geometry and configured into a truncated hexahedron shape.

2. A feminine hygiene apparatus of claim 1, where said absorbent material having substantially parallelepiped geometry is a truncated rectangular prism shape.

3. A feminine hygiene apparatus of claim 1, wherein said absorbent material is a plant material.

4. A feminine hygiene apparatus of claim 1, wherein said absorbent material is a sea sponge.

5. A feminine hygiene apparatus of claim 1, wherein said absorbent material is a Caribbean silk sea sponge.

6. A feminine hygiene apparatus of claim 1, wherein said absorbent material is a Jade sea sponge.

7. A feminine hygiene apparatus of claim 1, wherein said absorbent material is a Pearl sea sponge.

8. A feminine hygiene apparatus of claim 1, wherein said absorbent material is a Sheep's wool sea sponge.

9. A feminine hygiene apparatus of claim 1, wherein said absorbent material is a Bahama silk sea sponge.

10. A feminine hygiene apparatus of claim 1, wherein said absorbent material is 2.25 inches in length, 1 inch in width, and 1 inch in height.

11. A feminine hygiene apparatus of claim 1, wherein said apparatus is washable.

12. A feminine hygiene apparatus of claim 1, wherein said apparatus is reusable.

13. A feminine hygiene apparatus of claim 1, wherein said apparatus is configured to allow sexual intercourse.

14. A feminine hygiene system comprising:
at least two natural absorbent materials having substantially parallelepiped geometry and configured into a truncated hexahedron shape, wherein said absorbent materials are configured to each be positioned into a vaginal fornix within a vaginal cavity; and
said absorbent materials are configured to substantially block uterine fluid from leaving the vaginal cavity.

15. A feminine hygiene kit comprising:
a written set of instructions;
at least two natural absorbent materials having substantially parallelepiped geometry and configured into a truncated hexahedron shape, wherein said absorbent materials are each configured to be positioned into a vaginal fornix; and
a vacuum sealable container; wherein said natural absorbent materials are sealed inside said bag and said written set of instructions located on the outside of said container.
